(19) United States
(12) Patent Application Publication
(10) Pub. No.: US 2024/0200548 A1
(43) Pub. Date: Jun. 20, 2024

(54) TUBE WITH EMBEDDED MAGNETIC PARTICLES AND ASSOCIATED PUMPING DEVICE

(71) Applicant: Trelleborg Sealing Solutions Germany GmbH, Stuttgart (DE)

(72) Inventor: Larry Castleman, Monroeville, IN (US)

(73) Assignee: Trelleborg Sealing Solutions Germany GmbH, Stuttgart (DE)

(21) Appl. No.: 18/067,923

(22) Filed: Dec. 19, 2022

(51) Int. Cl. F04B 43/09; F04B 43/08

(52) U.S. Cl. CPC F04B 43/09; F04B 43/086; F04B 2203/04

(57) ABSTRACT

A pumping assembly includes a flexible tube extending along a longitudinal axis. The flexible tube defines an internal cavity extending along the longitudinal axis. The internal cavity is configured to contain a fluid disposed therein, wherein the fluid is a liquid and/or a gas. A plurality of ferromagnetic or magnetic particles are disposed within at least a portion of a thickness of the flexible tube. The plurality of ferromagnetic or magnetic particles are also disposed extending along the longitudinal axis. A plurality of electromagnets are placed in series adjacent to one another. The flexible tube is disposed adjacent to the plurality of electromagnets. A controller is configured to selectively magnetize at least one electromagnet from the plurality of electromagnets in a progression along the plurality of electromagnets, thereby compressing the tubing in a progression being a pumping action. A power source is electrically connected to the controller.

### CROSS-REFERENCE TO RELATED APPLICATIONS

None.

### DESCRIPTION

### Field of the Invention

The present invention generally relates to tubes used in the medical industry. More particularly, the present invention relates to a new tube design utilizing ferromagnetic and/or magnetic particles embedded therein and an associated pumping device that moves fluid disposed within the tube.

### Background of the Invention

The inventor's employer manufactures a complete line of medical and food grade silicone products for the life sciences and processing industries that meet U.S. Pharmacopeia (USP) Class VI standards. These include ultra-pure silicone hose and tubing (standard, custom, peristaltic pump, color-stripe, braided reinforced, platinum and peroxide) and reusable fittings, as well as validation assist devices, extruded profiles and overmolded assemblies. These silicone products are compatible with the extreme temperatures, steam and aggressive chemicals associated with Sterilization-in-Place (SIP) and Clean-in-Place (CIP) regimes. Applications supported include feeding pumps, drug-dispensing equipment, catheters, diagnostic equipment, disposable fluid transfer manifolds and closure assemblies, filtration gaskets, dialysis machines and processing equipment.

Many flow control systems in healthcare, medical, and other fields incorporate flexible tubing, pumps, valves and other elements to achieve the desired flow behavior. Often these systems have needs around sterility, cleanliness, leakage performance, precise flow rates in different channels, and other performance behaviors that make the installation, use and maintenance of these systems difficult. Separate pumps, valves, connectors, and other elements all have to be assembled together correctly, and then synchronized and maintained to continue to deliver proper performance. Providing methods to combine, eliminate, or replace elements with to provide better performance and end user experience would be beneficial.

Silicone tubing is widely used in the medical field with a peristaltic pump to deliver a flow of drugs, saline, blood, and other various fluids. The peristaltic pump, also commonly known as a roller pump, is a type of positive displacement pump used for pumping a variety of fluids. The fluid is contained in a flexible tube fitted inside a circular pump casing. Most peristaltic pumps work through rotary motion, though linear peristaltic pumps have also been made. The rotor has a number of “wipers” or “rollers” attached to its external circumference, which compress the flexible tube as they rotate by. The part of the tube under compression is closed, forcing the fluid to move through the tube. Additionally, as the tube opens to its natural state after the rollers pass, more fluid is drawn into the tube. This process is called peristalsis and is used in many biological systems such as the gastrointestinal tract. Typically, there will be two or more rollers compressing the tube, trapping a body of fluid between them. The body of fluid is transported through the tube, toward the pump outlet. Peristaltic pumps may run continuously, or they may be indexed through partial revolutions to deliver smaller amounts of fluid.

In addition to being used with a pumping system, the tubing system often have flow control valves that stop or regulate the pressure or flow of the fluid within the tube. As each junction in these tubing systems has a critical need to provide flow control and no leakage over long life, the addition of valves, Tee's and Y's creates solutions like overmolding, insertion, and other techniques that increase risk of leakage or contamination, or add sufficient complexity to the system.

Undesirably, the peristaltic pump wears down the silicone tubing it is engaging with, leading to cracks in the silicone tubing or the need to replace the tubing more frequently than desired. Additional undesirable features include: needing joints that are possible leakage and contamination points; noisy operation; surging or uneven flow due to the peristaltic pump roller setup; inefficient operation due to the aging/compression set of the cross section, which reduces flow over time and leads to short life; potentially large size of the pumping unit; additional sensing required to insure correct flow; less environmentally friendly given the high amount of things to dispose or clean after use; less flexibility in routing layouts for tubes and hoses; inability to tailor the dampening of pressure shock; more difficult to provide a failsafe (e.g., tube is automatically closed off when power not available); and more heat imparted into the fluid being pumped.

By using the technology of the present invention, it is possible to make a significantly easier process with no potential leak paths in the flow paths and more easily configurable flow/pressure control via placement of the magnets. Accordingly, there is a need for a better way to pump such critical fluids. The present invention fulfills these needs and provides other related advantages.

### SUMMARY OF THE INVENTION

An exemplary embodiment of the present invention is a pumping assembly, comprising: a flexible tube extending along a longitudinal axis; wherein the flexible tube defines an internal cavity extending along the longitudinal axis, the internal cavity configured to contain a fluid disposed therein, wherein the fluid comprises a liquid and/or a gas; a plurality of ferromagnetic or magnetic particles disposed within at least a portion of a thickness of the flexible tube, the thickness defined between an inner surface and an outer surface of the flexible tube, and wherein the plurality of ferromagnetic or magnetic particles are also disposed extending along the longitudinal axis; a plurality of electromagnets placed in series adjacent to one another; wherein the flexible tube is disposed adjacent to the plurality of electromagnets, where the longitudinal axis of the flexible tube is parallel with the plurality of electromagnets placed in series; a controller configured to selectively magnetize at least one electromagnet from the plurality of electromagnets in a progression along the plurality of electromagnets; and a power source electrically connected to the controller.

In other exemplary embodiments, while the flexible tube is disposed adjacent to the plurality of electromagnets, the at least a portion of the thickness of the flexible tube comprising the plurality of ferromagnetic or magnetic particles is oriented furthest away from the plurality of electromagnets, such that flexible tube compression is obtained during the magnetization of at least one electromagnet from the plurality of electromagnets.

In other exemplary embodiments, the plurality of ferromagnetic or magnetic particles may be evenly distributed throughout an entire circumferential thickness of the flexible tube. Alternatively, the plurality of ferromagnetic or magnetic particles may not be evenly distributed throughout an entire circumferential thickness of the flexible tube.

In other exemplary embodiments, a cross section taken perpendicular to the longitudinal axis through the flexible tube may define a top portion opposite a bottom portion and a left-side portion opposite a right-side portion of the flexible tube, wherein the bottom portion of the flexible tube is disposed nearest the plurality of the electromagnets.

The plurality of ferromagnetic or magnetic particles may be disposed only within the top portion of the flexible tube.

The plurality of ferromagnetic or magnetic particles may be disposed only within the top portion and bottom portion of the flexible tube and not disposed within the left-side portion or right-side portion.

The plurality of ferromagnetic or magnetic particles may be disposed in a higher density in the top portion and bottom portion in comparison to being disposed in the left-side portion and right-side portion.

The bottom portion of the tube may comprise a protrusion extending along the longitudinal axis, wherein the plurality of the electromagnets comprises a channel extending along the longitudinal axis, where the protrusion of the flexible tube is configured to nest within the channel of the plurality of the electromagnets. The protrusion may comprise a bulbus end that matches the channel it fits within.

A thickness of the flexible tube along the left-side portion and right-side portion may be thinner in comparison to a thickness along the bottom portion and top portion.

The cross section of the flexible tube may be circular or oval, rectangular or square, or hexagonal.

The plurality of ferromagnetic or magnetic particles may be disposed within a gel layer that is disposed within the at least a portion of the thickness of the flexible tube.

A left-side plurality of electromagnets and a right-side plurality of electromagnets may be disposed about the plurality of electromagnets, wherein the controller is configured to selectively magnetize at least one electromagnet from each of the left-side plurality of electromagnets, the right-side plurality of electromagnets and the plurality of electromagnets in a progression.

The flexible tube may further define a second internal cavity extending along the longitudinal axis that is not in fluidic communication with the internal cavity, wherein the plurality of ferromagnetic or magnetic particles are not disposed within a thickness surrounding the second internal cavity.

The flexible tube may comprise silicone, fluoropolymer, fluorosilicone, polyethylene, PVC, polyurethane, ethylene propylene, fluoroelastomer, latex or thermoplastic elastomers.

The power source may comprise a battery or an electrical plug.

A housing may be attached relative to the plurality of electromagnets and configured to secure the flexible tube in position against the plurality of electromagnets.

Other features and advantages of the present invention will become apparent from the following more detailed description, when taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is an isometric view of an exemplary embodiment of the present invention;

FIG. 2A is a sectional view taken along lines 2-2 of FIG. 1 showing an embodiment of the present invention;

FIG. 2B is the structure of FIG. 2A now showing the compression of the tube through electromagnetic attraction;

FIG. 3A is a sectional view taken along lines 3-3 of FIG. 1 showing another embodiment of the present invention;

FIG. 3B is the structure of FIG. 3A now showing the compression of the tube through electromagnetic attraction;

FIG. 4A is a sectional view taken along lines 4-4 of FIG. 1 showing another embodiment of the present invention;

FIG. 4B is the structure of FIG. 4A now showing the compression of the tube through electromagnetic attraction;

FIG. 5A is a sectional view taken along lines 5-5 of FIG. 1 showing another embodiment of the present invention;

FIG. 5B is the structure of FIG. 5A now showing the compression of the tube through electromagnetic attraction;

FIG. 6A is a sectional view taken along lines 6-6 of FIG. 1 showing another embodiment of the present invention;

FIG. 6B is the structure of FIG. 6A now showing the compression of the tube through electromagnetic attraction;

FIG. 7A is a sectional view taken along lines 7-7 of FIG. 1 showing another embodiment of the present invention;

FIG. 7B is the structure of FIG. 7A now showing the compression of the tube through electromagnetic attraction;

FIG. 8A is a sectional view taken along lines 8-8 of FIG. 1 showing another embodiment of the present invention;

FIG. 8B is the structure of FIG. 8A now showing the compression of the tube through electromagnetic attraction;

FIG. 9A is a sectional view taken along lines 9-9 of FIG. 1 showing another embodiment of the present invention;

FIG. 9B is the structure of FIG. 9A now showing the compression of the tube through electromagnetic attraction;

FIG. 10A is a sectional view taken along lines 10-10 of FIG. 1 showing another embodiment of the present invention;

FIG. 10B is the structure of FIG. 10A now showing the compression of the tube through electromagnetic attraction;

FIG. 11A is a sectional view taken along lines 11-11 of FIG. 1 showing another embodiment of the present invention;

FIG. 11B is the structure of FIG. 11A now showing the compression of the tube through electromagnetic attraction;

FIG. 12A is a sectional view taken along lines 12-12 of FIG. 1 showing another embodiment of the present invention;

FIG. 12B is the structure of 12 FIG. A now showing the compression of the tube through electromagnetic attraction;

FIG. 13A is a sectional view taken along lines 13-13 of FIG. 1 showing another embodiment of the present invention;

FIG. 13B is the structure of FIG. 13A now showing the compression of the tube through electromagnetic attraction;

FIG. 14A is a sectional view taken along lines 14-14 of FIG. 1 showing another embodiment of the present invention;

FIG. 14B is the structure of FIG. 14A now showing the compression of the tube through electromagnetic attraction;

FIG. 15A is a sectional view taken along lines 15-15 of FIG. 1 showing another embodiment of the present invention;

FIG. 15B is the structure of FIG. 15A now showing the compression of the tube through electromagnetic attraction;

FIG. 16A is a sectional view taken along lines 16-16 of FIG. 1 showing another embodiment of the present invention;

FIG. 16B is the structure of FIG. 16A now showing the compression of the tube through electromagnetic attraction;

FIG. 17A is a sectional view taken along lines 17-17 of FIG. 1 now showing the structure of FIG. 2 and depicting the movement of fluid inside the tube;

FIG. 17B is a sectional view of the structure of FIG. 17A now depicting the further movement of the fluid inside the tube from FIG. 17A;

FIG. 17C is a sectional view of the structure of FIG. 17A now depicting the further movement of the fluid inside the tube from FIG. 17B;

FIG. 18 is a sectional view similar to FIGS. 2-12 now depicting a housing securing the flexible tube in relation to the electromagnets;

FIG. 19 is a top sectional view of an embodiment of a tube design utilizing the present invention;

FIG. 20 is a top sectional view of another embodiment of a tube design utilizing the present invention; and

FIG. 21 is a top sectional view of another embodiment of a tube design utilizing the present invention now having a distribution manifold.

### DETAILED DESCRIPTION OF THE PREFERRED EMBODIMENTS

This application illustrates and discusses many embodiments of the present invention. It is understood by those skilled in the art that any one feature of a certain embodiment could be applied to a different embodiment taught herein, as this teaching is not intended to limit the possible embodiments to just those shown and described herein.

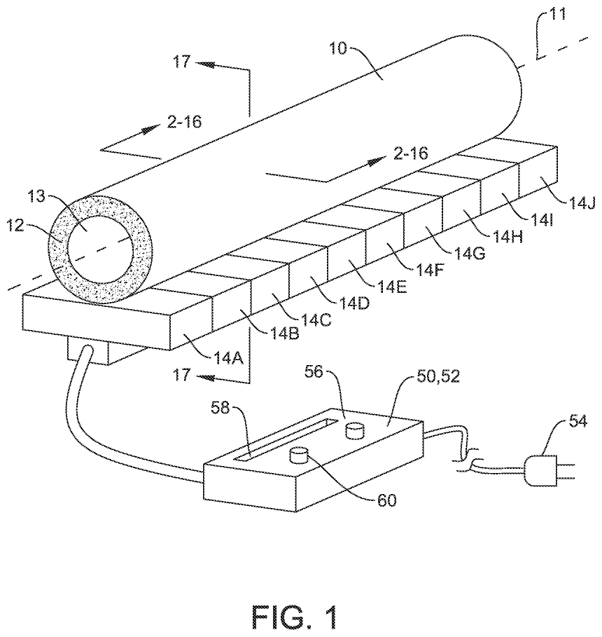

FIG. 1 is an isometric view of an embodiment of the present invention. A tube 10 has a multitude of embedded particles 12. The tube 10 extends along a longitudinal axis 11 and embodies a cavity 13 that also extends along the longitudinal axis. The tube can be silicone or other materials, such as fluoropolymer, fluorosilicone, polyethylene, PVC, polyurethane, ethylene propylene, fluoroelastomer, latex, thermoplastic elastomers, and other plastic and elastomer materials.

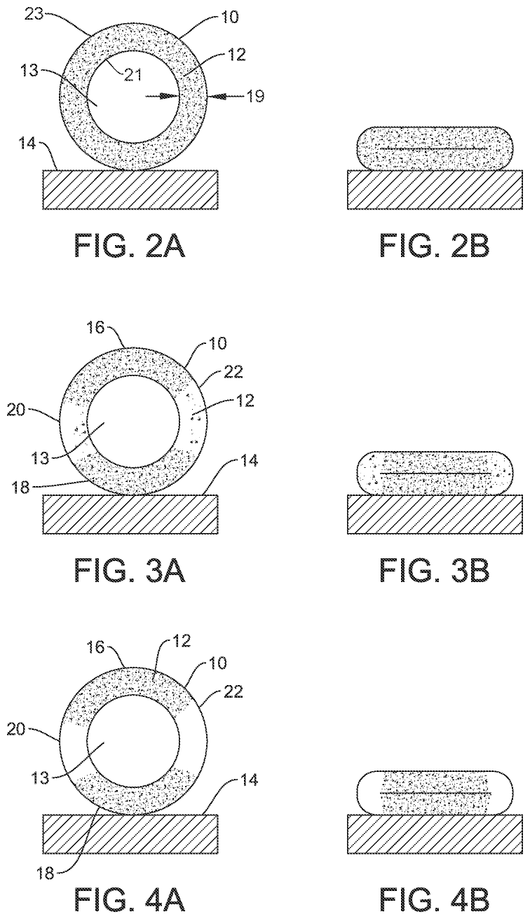

The plurality of ferromagnetic or magnetic particles are disposed within at least a portion of a thickness 19 of the flexible tube, where the thickness is defined between an inner diameter/surface 21 and an outer diameter/surface 23 of the flexible tube as best seen in FIG. 2A. It is also understood that the plurality of ferromagnetic or magnetic particles are disposed extending along the longitudinal axis such that the particles are disposed along the length of the tube. Said differently, the cavity 13 is defined by the inner surface 21 as it extends along the longitudinal axis 11.

The embedded particles may be magnetic or ferromagnetic. The embedded particles may also be magnetic nanoparticles, which are a class of nanoparticle that can be manipulated using magnetic fields. Such particles commonly consist of two components, a magnetic material, often iron, nickel and cobalt, and a chemical component that has functionality. While nanoparticles are smaller than 1 micrometer in diameter (typically 1-100 nanometers), the larger microbeads are 0.5-500 micrometer in diameter. Magnetic nanoparticle clusters that are composed of a number of individual magnetic nanoparticles are known as magnetic nanobeads with a diameter of 50-200 nanometers. Magnetic nanoparticle clusters are a basis for their further magnetic assembly into magnetic nanochains. Iron, nickel, cobalt, chromium, manganese, gadolinium, and their chemical compounds like ferrites, may be many times coated to insure compatibility/bondibility/solubility within or with another material.

A series of electromagnets 14A-14J are disposed in a line, such that the tube can be placed against the series of electromagnets. The electromagnets may be made from ferromagnetic materials or rare earth magnets such as neodymium and samarium cobalt. It is understood by those skilled in the art that two to any number “n” of electromagnets 14 may be disposed in a linear array, as this embodiment is just one example of many.

FIG. 2A is a sectional view taken along lines 2-2 of FIG. 1 showing an embodiment of the present invention. Fluid, such as a gas or liquid would reside in the cavity 13. FIG. 2B is the structure of FIG. 2A now showing the compression of the tube through electromagnetic attraction when the magnet is turned on. In this embodiment, the particles are evenly distributed throughout the entire circumference of the tube. In FIG. 2B, the magnet 14 is attracting the particles 12 such that the tube is forced closed.

FIG. 3A is a sectional view taken along lines 3-3 of FIG. 1 showing another embodiment of the present invention. FIG. 3B is the structure of FIG. 3A now showing the compression of the tube through electromagnetic attraction when the magnet is turned on. In this embodiment, the particles are not evenly distributed. Rather, the particles at the top 16 and bottom 18 of the tube are more dense in comparison the particles at the left 20 and right 22 of the tube. When the electromagnet is turned on this would create a stronger magnetic attraction force at the top and bottom for an improved pumping affect.

FIG. 4A is a sectional view taken along lines 4-4 of FIG. 1 showing another embodiment of the present invention. FIG. 4B is the structure of FIG. 4A now showing the compression of the tube through electromagnetic attraction when the magnet is turned on. In this embodiment, the particles are only disposed at the top 16 and bottom 18 of the tube and not at the left 20 and right 22 sides of the tube.

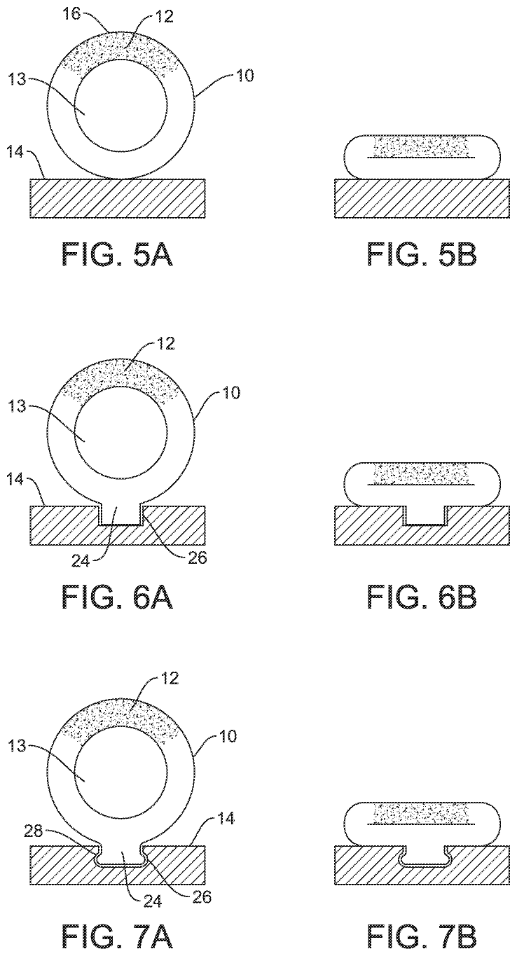

FIG. 5A is a sectional view taken along lines 5-5 of FIG. 1 showing another embodiment of the present invention. FIG. 5B is the structure of FIG. 5A now showing the compression of the tube through electromagnetic attraction when the magnet is turned on. In this embodiment, the particles are only disposed at the top 16 of the tube. As is shown in FIG. 5B, the particles would still be attracted to the electromagnet when it is turned on such that the pumping affect would still be possible.

FIG. 6A is a sectional view taken along lines 6-6 of FIG. 1 showing another embodiment of the present invention. FIG. 6B is the structure of FIG. 6A now showing the compression of the tube through electromagnetic attraction when the magnet is turned on. In this embodiment, the tube has a protrusion 24. The protrusion is then designed to nest within a channel 26 formed in the electromagnets. In this manner, the tube and magnets are able to index relative to one another, such that tube twisting can be prevented or eliminated.

FIG. 7A is a sectional view taken along lines 7-7 of FIG. 1 showing another embodiment of the present invention. FIG. 7B is the structure of FIG. 7A now showing the compression of the tube through electromagnetic attraction when the magnet is turned on. This embodiment is similar to FIG. 6, but now the protrusion 24 has a bulbus end 28 that matches the channel 26 it fits within. In this manner, the protrusion can be press fit into the channel and create a locking action that helps to secure the protrusion within the channel.

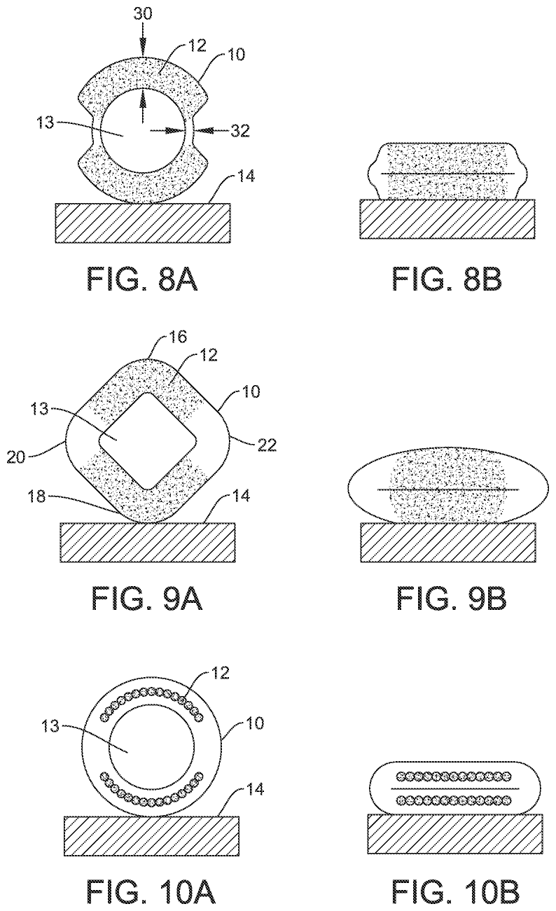

FIG. 8A is a sectional view taken along lines 8-8 of FIG. 1 showing another embodiment of the present invention. FIG. 8B is the structure of FIG. 8A now showing the compression of the tube through electromagnetic attraction when the magnet is turned on. In this embodiment the particles are only disposed at the top and bottom of the tube. Now, the sides of the tube are smaller in thickness 32 in comparison to the thickness 30 at the top and bottom. This would naturally create a flexural joint at the left and right sides such that compression of the tube is facilitated. This could lead to a better closure as shown in FIG. 8B.

FIG. 9A is a sectional view taken along lines 9-9 of FIG. 1 showing another embodiment of the present invention. FIG. 9B is the structure of FIG. 9A now showing the compression of the tube through electromagnetic attraction when the magnet is turned on. In this embodiment, the tube is rectangular or square in shape and not circular as shown in FIGS. 2-8. Here, the particles are once again disposed at the top 16 and bottom 18 and not at the left 20 and right 22 sides.

FIG. 10A is a sectional view taken along lines 10-10 of FIG. 1 showing another embodiment of the present invention. FIG. 10B is the structure of FIG. 10A now showing the compression of the tube through electromagnetic attraction when the magnet is turned on. In this embodiment, the particles are not randomly disposed throughout the thickness but instead radially centered in series along the top and the bottom of the tube. As shown here, there is one layer of particles being aligned. However, it is understood that 1, 2, 3 or any number “n” of concentrically disposed array of particles could be used by those skilled in the art.

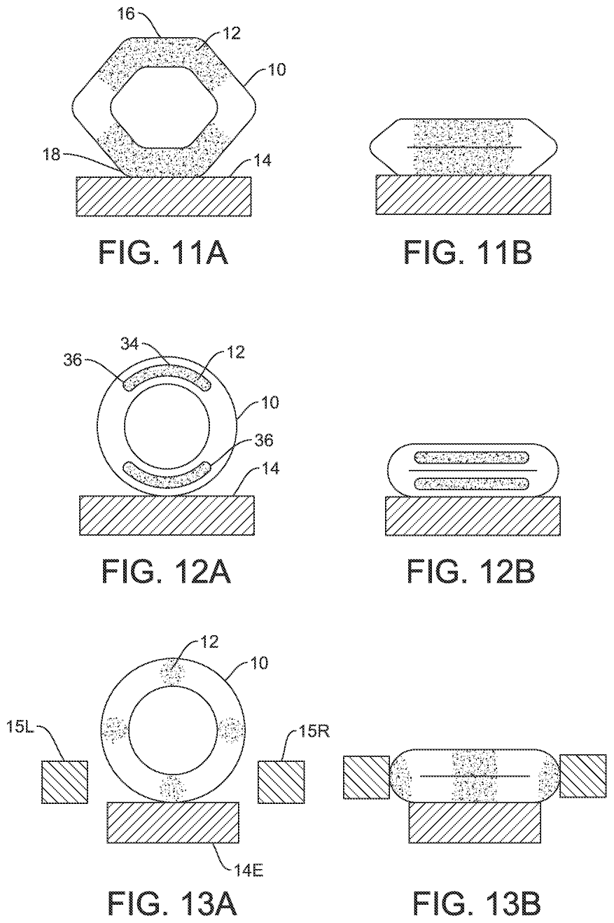

FIG. 11A is a sectional view taken along lines 11-11 of FIG. 1 showing another embodiment of the present invention. FIG. 11B is the structure of FIG. 11A now showing the compression of the tube through electromagnetic attraction when the magnet is turned on. In this embodiment, the sectional shape is hexagonal. The particles are shown disposed at the top 16 and bottom 18 but could be disposed as is shown in any of the other embodiments.

FIG. 12A is a sectional view taken along lines 12-12 of FIG. 1 showing another embodiment of the present invention. FIG. 12B is the structure of FIG. 12A now showing the compression of the tube through electromagnetic attraction when the magnet is turned on. In this embodiment, a gel layer 34 contains the particles 12 disposed therein. The tube could be formed with a top and/or bottom cavity 36 such that the gel is later disposed therein. Alternatively, the gel layer 34 may be disposed at the time of extrusion of the tube 10.

FIG. 13A is a sectional view taken along lines 13-13 of FIG. 1 showing another embodiment of the present invention. FIG. 13B is the structure of FIG. 13A now showing the compression of the tube through electromagnetic attraction when the magnet is turned on. In this embodiment, particles are disposed selectively at just the top, bottom, left and right of the tube. Then, a left-side electromagnet 15L and a right-side electromagnet 15R help pull the left and right side ends apart while the center electromagnet 14E (i.e., 14A-J) still pulls the top and bottom sides downward. As can be appreciated, the tube is being compressed and pulled at the same time. This could lead to an improved seal and pumping action.

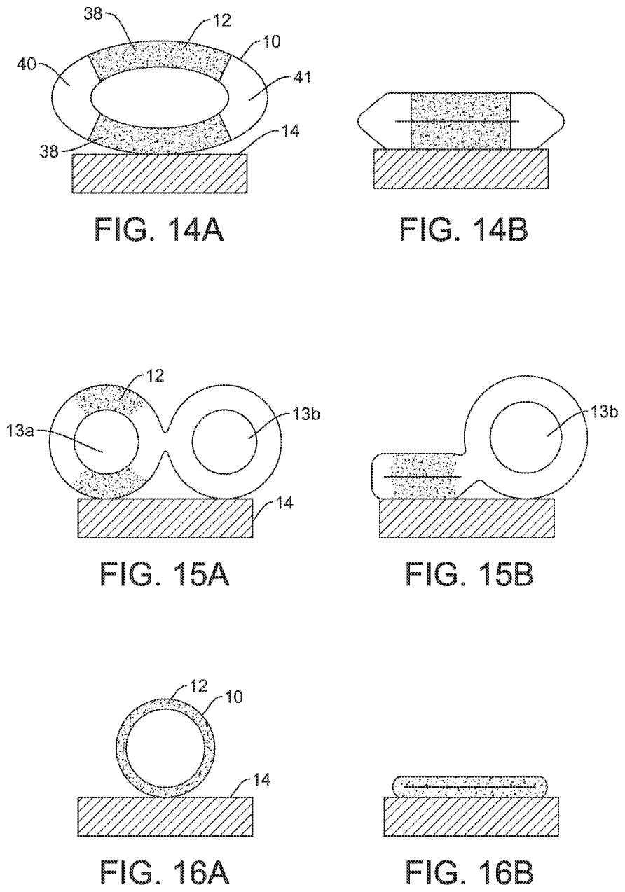

FIG. 14A is a sectional view taken along lines 14-14 of FIG. 1 showing another embodiment of the present invention. FIG. 14B is the structure of FIG. 14A now showing the compression of the tube through electromagnetic attraction when the magnet is turned on. In this embodiment, the top and bottom portions 38 could be made of a thicker density of silicone tubing, such that the elastic modulus was higher in comparison to the left 40 and right 41 sides. Thus, the portions 38 would be stiffer in comparison to the portions 40.

FIG. 15A is a sectional view taken along lines 15-15 of FIG. 1 showing another embodiment of the present invention. FIG. 15B is the structure of FIG. 15A now showing the compression of the tube through electromagnetic attraction when the magnet is turned on. In this embodiment, the tube 12 is a multi-lumen tube. Here, there are two cavities 13a and 13b. The particles 12 are disposed only around the first cavity 13a. This means that the cavity 13a can be used for pumping while cavity 13b can be used to contain electrical wires, facilitate drainage or a vacuum connection. It will be understood that any number of cavities could be used from 2 to any “n” number of cavities in a multi-lumen tube. Additionally, the magnetic particles 12 may be disposed around 1 or any “n” number of cavities.

FIG. 16A is a sectional view taken along lines 16-16 of FIG. 1 showing another embodiment of the present invention. FIG. 16B is the structure of FIG. 16A now showing the compression of the tube through electromagnetic attraction when the magnet is turned on. In this embodiment, a much thinner tubing 12 is used such that it would have a greater ability to collapse and expand, thus changing the flow rate in comparison to other embodiments shown herein. It is understood by those skilled in the art that a wide variety of tubing sizes, thicknesses and shapes could be used as this teaching is not limited to just the precise embodiments shown herein.

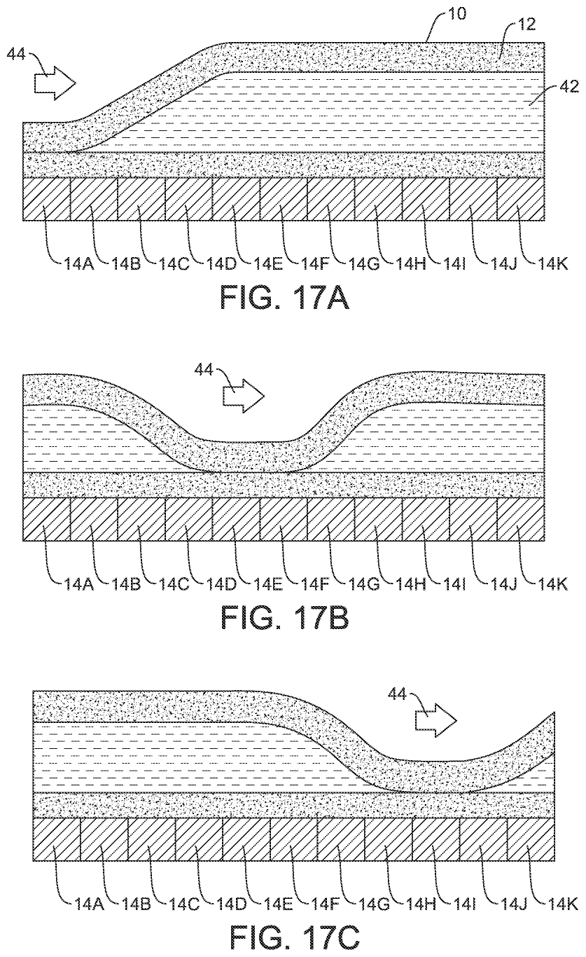

FIG. 17A is a sectional view taken along lines 17-17 of FIG. 1 now showing the structure of FIG. 2 and depicting the movement of fluid inside the tube. A series of electromagnets 14A-14K are disposed left to right. The tube 10 sits directly against the series of electromagnets 14. In this figure, the electromagnet for 14A is turned on and other electromagnets are turned off. The movement of the fluid flow is depicted as arrow 44.

FIG. 17B is a sectional view of the structure of FIG. 17A now depicting the further movement of the fluid inside the tube from FIG. 17A. Now, the electromagnetic 14E is turned on and the other electromagnetics are turned off. It is understood by those skilled in the art that the progression of electromagnets being turned on and off would start with 14A, then to 14B, then to 14C and so on. In this manner, the seal 42 being formed is maintained through the progression of electromagnets.

FIG. 17C is a sectional view of the structure of FIG. 17A now depicting the further movement of the fluid inside the tube from FIG. 17B. Now, the electromagnet 141 is turned on and the other electromagnets are turned off. The movement 44 of the progression and fluid can be understood by those skilled in the art. Furthermore, it is understood that while in this embodiment just one electromagnet is turned on and off, that two, three or any number of adjacent electromagnets may be used to facilitate the pumping action and movement 44.

Referring back to FIG. 1, the plurality of electrode magnets is controlled by a controller 50. The controller 50 is powered either by an internal battery 52 or is plugged into a wall socket via a power cable 54. A control panel 56 has a display 58 and any number of buttons or controls 60. This teaching is not limited to the precise form described herein, but it is understood by those skilled in the art that the controller 50 may take on any number of differing embodiments as this example is just one example of such an embodiment.

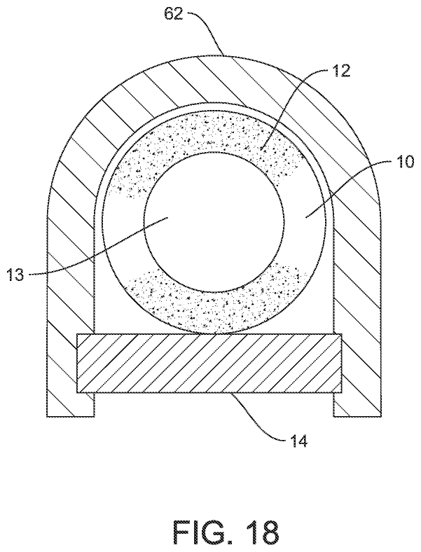

FIG. 18 is a sectional view similar to FIGS. 2-12 now depicting a housing 62 securing the flexible tube in relation to the electromagnets. The housing 62 can take on many shapes and sizes, as its function is to hold the flexible tube 10 in location so that it remains adjacent the electromagnets 14. Thus, the housing 62 can be permanent or removable, such that replacing the tubing within is easy.

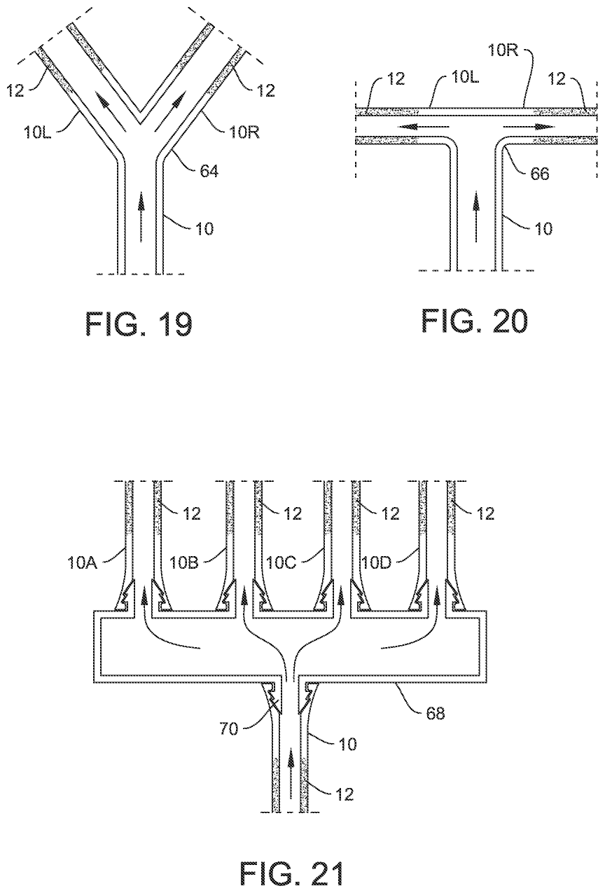

FIG. 19 shows a top sectional view of another embodiment of a tube 10 that has a Y-shaped portion 64 splitting off into a left tube 10L and a right tube 10R. The embedded particles 12 may be placed into each left and right tube portions such that different flow rates may be achieved despite being fed from the same common portion. Here, the arrows represent the flow direction of the fluid inside the tubes. It is understood that the electromagnets 14 would be appropriately placed adjacent to the tubes 10L and 10R to create a progression of compression that facilitates fluid movement.

FIG. 20 shows a top sectional view of yet another embodiment of a tube 10 that has a T-shaped portion 66 splitting off into a left tube 10L and a right tube 10R. Similar to FIG. 19, the embedded particles 12 may be placed into each left and right tube portions such that different flow rates may be achieved despite being fed from the same common portion. Here, the arrows represent the flow direction of the fluid inside the tubes. It is understood that the electromagnets 14 would be appropriately placed adjacent to the tubes 10L and 10R to create a progression of compression that facilitates fluid movement.

FIG. 21 shows a top sectional view of yet another embodiment of the present invention where a distribution manifold 10 is used to create a plurality of additional tubes such as 2, 3, 4 or any “n” number of tubes. As shown here, there is a primary supply tube 10 that is connected to the manifold 68. Here, a barbed connection 70 is shown for ease of connection, but any known connection methods known by those skilled in the art could be used. Here, there are additional tubes 10A, 10B, 10C and 10D that are fed by the manifold 68. The embedded particles 12 may be placed into each additional tubes 10A, 10B, 10C and 10D such that different flow rates may be achieved despite being fed from the same common portion. Here, the arrows represent the flow direction of the fluid inside the tubes. It is understood that the electromagnets 14 would be appropriately placed adjacent to the tubes 10L and 10R to create a progression of compression that facilitates fluid movement. Optionally, as shown, even the supply tube 10 can have its own embedded particles 12 and have its own fluid flow rate that supplies the manifold 68. As can be appreciated in light of FIGS. 19-21, those skilled in the art may create a multitude of tube designs that utilize the present invention as these few teachings are not exhaustive of the possible combinations.

The invention scope includes nano or micro particles embedded with the tube and hose structure such that upon an external field (magnetic field, other forms of excitement) would cause an attraction to expand or collapse the hose as required, and to provide sensing of the flow, viscosity, or other condition of the flow stream. Existing solutions using involve pumps (gear, peristaltic, piston, etc.). Useful advantages depending on application:

The present invention may have the following advantages over the prior art: non-contact to the fluid; longer life for the tube with less stress/strain than peristaltic pump; better flow control (better accuracy for high range of low to high volume pumping); quieter; more compact; includes built in sensing; more environmentally friendly (less disposable elements in landfill or recycling than other system layouts); unique layouts (tight corners, etc.); ability to dampen pressure shocks better; ability to have fail safe (ex. tube is automatically closed off when electricity not present); more energy efficient; less heat.

Although several embodiments have been described in detail for purposes of illustration, various modifications may be made to each without departing from the scope and spirit of the invention. Accordingly, the invention is not to be limited, except as by the appended claims.

What is claimed is:

1. A pumping assembly, comprising:
a flexible tube extending along a longitudinal axis;
wherein the flexible tube defines an internal cavity extending along the longitudinal axis, the internal cavity configured to contain a fluid disposed therein, wherein the fluid comprises a liquid and/or a gas;
a plurality of ferromagnetic or magnetic particles disposed within at least a portion of a thickness of the flexible tube, the thickness defined between an inner surface and an outer surface of the flexible tube, and wherein the plurality of ferromagnetic or magnetic particles are also disposed extending along the longitudinal axis;
a plurality of electromagnets placed in series adjacent to one another;
wherein the flexible tube is disposed adjacent to the plurality of electromagnets, where the longitudinal axis of the flexible tube is parallel with the plurality of electromagnets placed in series;
a controller connected to the plurality of electromagnets and configured to selectively magnetize at least one electromagnet from the plurality of electromagnets in a progression along the plurality of electromagnets; and
a power source electrically connected to the controller.

2. The pumping assembly of claim 1, wherein, while the flexible tube is disposed adjacent to the plurality of electromagnets, the at least a portion of the thickness of the flexible tube comprising the plurality of ferromagnetic or magnetic particles is oriented furthest away from the plurality of electromagnets, wherein flexible tube compression is obtained during the magnetization of at least one electromagnet from the plurality of electromagnets.

3. The pumping assembly of claim 1, wherein the plurality of ferromagnetic or magnetic particles are evenly distributed throughout an entire circumferential thickness of the flexible tube.

4. The pumping assembly of claim 1, wherein the plurality of ferromagnetic or magnetic particles are not evenly distributed throughout an entire circumferential thickness of the flexible tube.

5. The pumping assembly of claim 1, wherein a cross section taken perpendicular to the longitudinal axis through the flexible tube defines a top portion opposite a bottom portion and a left-side portion opposite a right-side portion of the flexible tube, wherein the bottom portion of the flexible tube is disposed nearest the plurality of the electromagnets.

6. The pumping assembly of claim 5, wherein the plurality of ferromagnetic or magnetic particles are disposed only within the top portion of the flexible tube.

7. The pumping assembly of claim 5, wherein the plurality of ferromagnetic or magnetic particles are disposed only within the top portion and bottom portion of the flexible tube and not disposed within the left-side portion or right-side portion.

8. The pumping assembly of claim 5, wherein the plurality of ferromagnetic or magnetic particles are disposed in a higher density in the top portion and bottom portion in comparison to being disposed in the left-side portion and right-side portion.

9. The pumping assembly of claim 5, wherein the bottom portion of the tube comprises a protrusion extending along the longitudinal axis, wherein the plurality of the electromagnets comprises a channel extending along the longitudinal axis, where the protrusion of the flexible tube is configured to nest within the channel of the plurality of the electromagnets.

10. The pumping assembly of claim 9, wherein the protrusion comprises a bulbus end that matches the channel it fits within.

11. The pumping assembly of claim 5, wherein a thickness of the flexible tube along the left-side portion and right-side portion is thinner in comparison to a thickness along the bottom portion and top portion.

12. The pumping assembly of claim 5, wherein the cross section is circular or oval.

13. The pumping assembly of claim 5, wherein the cross section is rectangular or square.

14. The pumping assembly of claim 5, wherein the cross section is hexagonal.

15. The pumping assembly of claim 1, wherein the plurality of ferromagnetic or magnetic particles are disposed within a gel layer that is disposed within the at least a portion of the thickness of the flexible tube.

16. The pumping assembly of claim 1, further including a left-side plurality of electromagnets and a right-side plurality of electromagnets disposed about the plurality of electromagnets, wherein the controller is configured to selectively magnetize at least one electromagnet from each of the left-side plurality of electromagnets, the right-side plurality of electromagnets and the plurality of electromagnets in a progression.

17. The pumping assembly of claim 1, wherein the flexible tube further defines a second internal cavity extending along the longitudinal axis that is not in fluidic communication with the internal cavity, wherein the plurality of ferromagnetic or magnetic particles are not disposed within a thickness surrounding the second internal cavity.

18. The pumping assembly of claim 1, wherein the flexible tube comprises silicone, fluoropolymer, fluorosilicone, polyethylene, PVC, polyurethane, ethylene propylene, fluoroelastomer, latex or thermoplastic elastomers.

19. The pumping assembly of claim 1, wherein the power source comprises a battery or an electrical plug.

20. The pumping assembly of claim 1, further including a housing attached relative to the plurality of electromagnets and configured to secure the flexible tube in position against the plurality of electromagnets.
